(19) United States
(12) Patent Application Publication
(10) Pub. No.: US 2019/0209347 A1
(43) Pub. Date: Jul. 11, 2019

(54) Synergetic Prosthesis

(71) Applicant: Ashvin Saikumar Irrinki, Cupertino, CA (US)

(72) Inventor: Ashvin Saikumar Irrinki, Cupertino, CA (US)

(21) Appl. No.: 15/796,871

(22) Filed: Jan. 9, 2018

(51) Int. Cl. A61F 2/68; A61F 2/64; A61F 2/66; A61B 5/00

(52) U.S. Cl. CPC A61F 2/68; A61F 2/64; A61F 2002/5081; A61B 5/4836; A61F 2/6607

(57) ABSTRACT

The synergetic prosthetic system comprises of a knee and an ankle sleeve on the functional leg and a prosthesis on the amputated leg. Each sleeve comprises of a power supply, an accelerometer, a wireless transmitter and a wire connecting the accelerometer to the wireless transmitter. The prosthesis comprises of a power supply, a microcontroller, servo motors, a knee joint assembly, and an ankle joint assembly. A steel rod covered with a casing connects the knee and ankle joints. The accelerometer measures and transmits the angle of rotation of the functional knee and ankle joint to a microcontroller. The microcontroller pre-programmed with normal gait data pairs the data from the healthy leg and controls the servo motors. The servo motors, move the prosthetic joints to mimic a normal human gait.

### CROSS REFERENCES TO RELATED APPLICATIONS

Not Applicable

### FEDERALLY SPONSORED RESEARCH OR DEVELOPMENT

Not Applicable

### BACKGROUND

### Field of the Invention

The present invention relates generally to a prosthetic device for single-leg above the knee amputees. More particularly, the synergetic prosthesis system utilizes signals from the position sensors on the healthy leg and preprogrammed gait data stored in a microcontroller to move the prosthesis in synergy to simulate near normal human gait.

### Description of Related Art

Over the years, humans with an inability to walk due to amputations relied on prosthetics to serve their most basic needs. Vital limbs, such as legs, now have prosthetics that can efficiently compensate for the lost limb. The first prosthetics were simply wooden pegs relying on limbs above the amputated region to move. Prosthetics have managed to escalate in design and functionality over time. Modern-day prosthetics advanced from simple wooden mechanism to intricate and elaborate designs that actually aid in the normal human gait. However, these prosthetics are solely responsible to provide comfort to the amputees, despite having to mimic something as intricate as the normal human gait. Current prosthetics rely on repetitive and simple mechanisms such as the use of momentum or hydraulics in order to propel the limb forward that try their best to replicate the normal human gait, but fait to do so. Others use motors, but receive information from the brain waves. Current prosthetics lack control from the user and prevent the amputees from normal functional gait pattern. In the prior art, the prosthetic industry is continuously making strides to mimic normal human functional gait pattern.

Based on recent studies, prosthetics that fail to comply with the intricacies of the normal human gait causes unwarranted problems, including back pain. Researchers have tackled this issue and were partially successful, as they created a prosthesis that harnesses brain waves from the patient in order to control the prosthetic. As promising as this prosthetic may be, the costs to obtain one is appalling. Although prosthetic design has advanced in recent years, the prior art still has limitations to meet the functional needs of the amputees. There exists an extensive opportunity for design advancements and innovation where the prior art fails or is deficient. It is therefore an object of the present invention to mitigate these hindrances.

### SUMMARY

In general, the present invention of a synergetic prosthesis for single-leg above the knee amputees provides normal human gait pattern where the prior art fails. The present invention generally will improve an amputees gait by receiving electronic signals from the healthy functional leg, compare the signals with pre-programmed gait analysis data and transmit signals to the prosthetic to allow the amputees gait to be reminiscent of a normal human gait. This prosthesis involves work in three major fields, the electrical, mechanical and the medical fields which contributes to its difficulty. The mechanical aspect involves the design and construction of a viable prosthetic. The electrical aspect involves the programming and the writing of the prosthetic. The medical aspect involves understanding the human gait study.

The human gait is the mapped motion of the precise way we walk. Dynamic gait evaluation provides intrinsic and extrinsic factors affecting an individual's ability to walk. The evaluation also provides range of motion or kinematics such as positions, angles, velocities, and accelerations of body segments and joints during motion. In addition, kinetics or forces that cause the body to move are evaluated using electromyogram (EMG) tracing of the muscle activity. This information is widely published and readily available for use in the design of the prosthetics. The present invention uses this data to move the prosthesis in synergy with the healthy leg to mimic the normal human gait.

Over the course of countless studies, scientists have narrowed an entire stride of human gait into two main phases, the Stance Phase and the Swing Phase. There are multiple sub phases in each main phase. The Stance Phase contributes to 60% of the stride which includes Initial Contract (heel strike), Loading Response (foot flat), Mid Stance, Terminal Stance (heel off), and Pre-Swing (toe-off). The Swing Phase contributes to 40% of the stride which includes Initial Swing (acceleration), Mid Swing, and Terminal Swing (deceleration). The prosthetics that do not follow the human gait have been proven to cause back and foot pain. In order to eliminate discomfort, the aim of this prosthetic is to model the human gait as precisely and accurately as possible. This prosthetic is strictly for patients who have a functional and healthy leg, but require a prosthesis for the other leg.

The key design of the synergetic prosthesis is to follow the human gait cycle by receiving and interpreting angle of rotation of the knee and ankle joints from the healthy leg and move the prosthetic to corresponding position to mimic the human gait. To achieve this, the use of accelerometers to properly measure the position is necessary. The accelerometers will be placed on the shin and the foot of the functional leg, to measure the knee and ankle joints, respectively. Two servo motors will be placed on the prosthetic knee and the ankle at the same position as the healthy leg to achieve the desired synergy. The accelerometers will measure the angle of rotation of the knee and ankle, and wirelessly send this data to the microcontroller. The microcontroller is pre-programmed with the normal gait cycle data to aid in the determination of the position of the prosthetic joints. For description purposes, in the normal human gait, the right knee angle is labelled ANGLE 1 and the left knee angle is labelled ANGLE 2. Similarly, the right ankle angle is labelled ANGLE 3, and the left ankle angle is labelled ANGLE 4. For instance, if the left leg is amputated, ANGLE 2 and 4 will be represented on the prosthetic leg. This means that the microcontroller will pair ANGLE 1 with ANGLE 2 and ANGLE 3 with ANGLE 4. In other words, if the accelerometer reads ANGLE 1 on the healthy knee, it will send the data to the micro-controller, which will pair it with ANGLE 2. On the prosthetic leg. Then, the microcontroller signals the servo motor to move to ANGLE 2. The servo motors will cause the prosthesis joints to move accordingly, synchronous to the healthy leg.

The results conclude that it is possible to use the joint angles of the healthy leg to move the prosthetic knee and ankle joints. Though not as appealing, the design and construction of this prosthetic allows the amputees to have proper mobility and comfort, as it follows human gait. This prosthetic can help thousands of people with lower limb amputations. By successfully improving the design and materials, this prosthetic will be very useful and aid many people.

The disclosed embodiments are illustrative, not restrictive. While specific configurations of the prosthetic have been described, it is understood that the present invention can be applied to a wide variety of prosthesis. There are many alternative ways of implementing the invention.

These and other features, aspects, and advantages of the present invention will become better understood with reference to the following description and claims.

### BRIEF DESCRIPTION OF THE DRAWINGS

Note: For simplicity of illustration, bearings, nuts, bolts, washers, and minutiae of common prosthesis industry hardware are not depicted as they are known to those with skill in the art. When they are shown, it is purely for illustrative purposes and not intended to capture all embodiments of the invention disclosed.

FIG. 1 is a perspective assembled view of the knee and ankle sleeves.

FIG. 2 is an exploded view of the knee sleeve shown in FIG. 1.

FIG. 3 is an exploded view of the ankle sleeve shown in FIG. 1.

FIG. 4 is a perspective assembled view of the synergetic prosthesis with the knee and the ankle joint

FIG. 5 is an exploded view of the prosthetic knee and the ankle joint shown in FIG. 4.

FIG. 6 is a block diagram showing an example of a control system for the synergetic prosthetic system as shown in FIG. 1 and FIG. 4.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

a. Accelerometer—a sensor that reads the angle of rotation while measuring the acceleration forces
b. Microcontroller—a control board that allows various types of input/output devices to interface with the board
c. Servo motor—is a rotary actuator or linear actuator that allows for precise control of angular or linear positions, velocity and acceleration
d. Synergy—“the interaction or cooperation of two or more organizations, substances, or other agents to produce a combined effect greater than the sum of their separate effects.”
e. Human Gait Cycle—the precise, mapped cycle of the way humans walk “Human gait refers to locomotion achieved through the movement of human limbs”

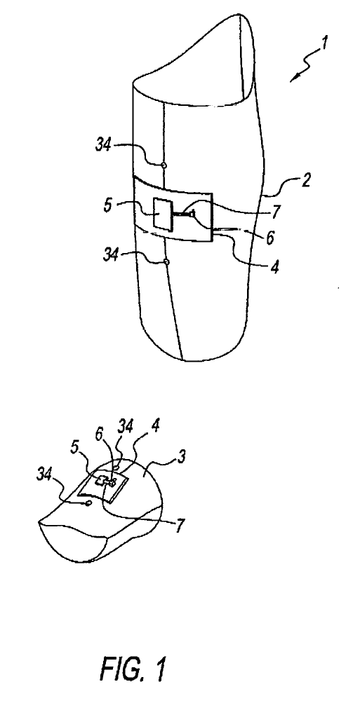

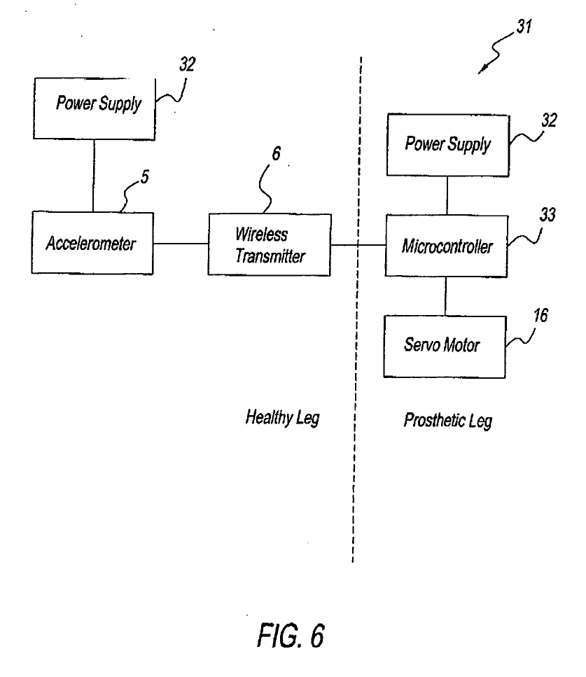

In FIG. 1, a perspective view of the knee and ankle sleeves (1) of the healthy leg are shown. A flexible, polyester knee sleeve (2) will slide over the patient's shin. An aluminum plate (4) is placed on the sleeve (2) to hold an accelerometer (5), and a wireless transmitter (6). A power supply (32), as shown in FIG. 6, supplies power to the accelerometer (5) and the wireless transmitter (6). The accelerometer (5) reads the angle of rotation when the knee is moving. To ensure the accelerometer (5) measure the same angles and readings for each stride the amputee takes, two positional markers (34) on the knee sleeve (2) are used to properly match with the position markers on the shin. A connecting wire (7) transfers the readings from the accelerometer to the wireless transmitter (6). The wireless transmitter (6) transmits the readings to the microcontroller (33), shown in FIG. 6. Another flexible, polyester foot sleeve (3) will slide over the patient's foot and has the same components and purpose as the knee sleeve (2). An aluminum plate (4) is placed on the foot sleeve (3) to hold an accelerometer (5), and a wireless transmitter (6). A power supply (32), as shown in FIG. 6, supplies power to the accelerometer (5) and the wireless transmitter (6). The accelerometer (5) reads the angle of rotation when the ankle is moving. To ensure the accelerometer (5) measures the same angles and readings for each stride the amputee takes, two positional markers (34) on the foot sleeve (2) are used to properly match with the position markers on the foot. The connecting wire (7) transfers the readings from the accelerometer to the wireless transmitter (6). The wireless transmitter (6) transmits the readings to the microcontroller (33), shown in FIG. 6.

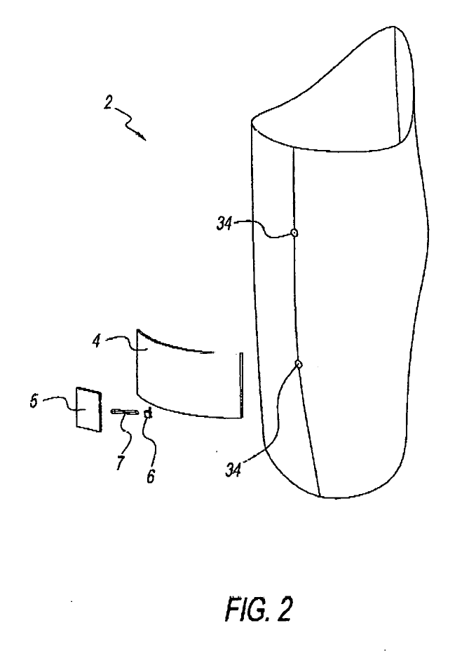

In FIG. 2, an exploded view of the knee sleeve (2) is shown. The accelerometer (5), the connecting wire (7), the wireless transmitter (6), the position markers (34), and the aluminum plate (4) can be seen in detail.

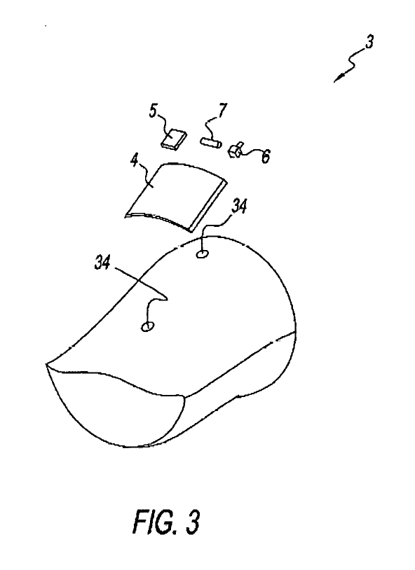

In FIG. 3, an exploded view of the ankle sleeve (3) is shown. The accelerometer (5), the connecting wire (7), the wireless transmitter (6), the position markers (34), and the aluminum plate (4) can be seen in detail.

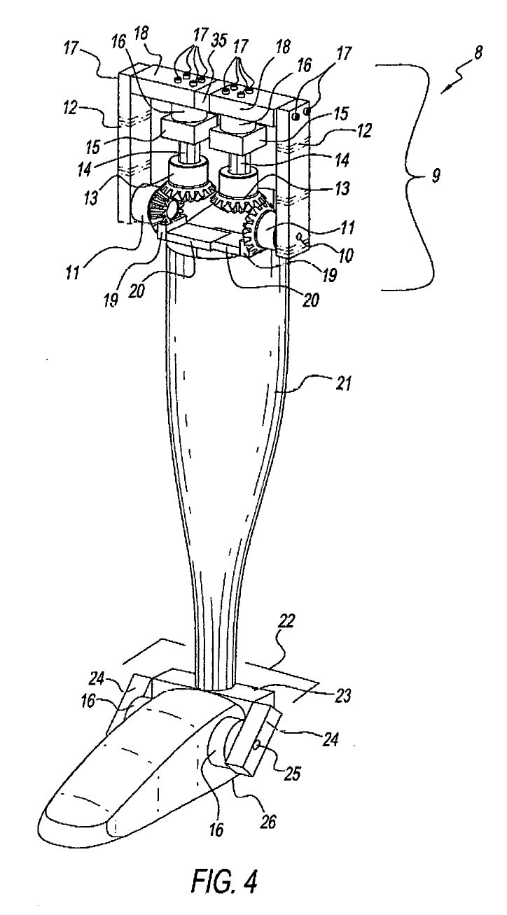

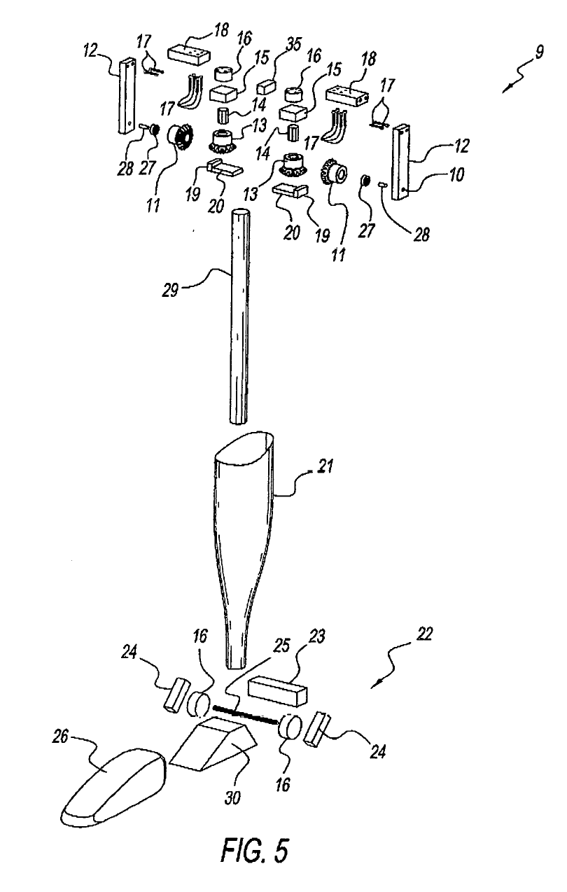

In FIG. 4, a perspective view of the synergetic prosthesis (8) is shown. The prosthetic knee joint (9) is responsible for moving the leg forwards and backwards. In order to do this, two servo motors (16) are connected. They are mounted by 4 screws (17) to a horizontal support member (18). The two servo motors (16) are placed vertically to conserve space in the vertical direction. As the two servo motors (16) need to move the entire leg, a gearbox (15) is needed to increase the torque the servo motor (16) produce. A metal shaft (14) is used to transfer the motion to an inverted gear (13). As the servo motors (16) is produce motion in the horizontal direction, a second gear (11) is needed to convert the horizontal motion to vertical motion. This gear (11) contains a steel hall bearing (27) for a smoother motion. A small, steel shaft (28), as shown in FIG. 5, is used to connect this gear (11) and the bearing (27) to a vertical support member (12). To secure this shaft (28), a sleeve is inserted into the vertical member (12). This vertical support member (12) is connected to the horizontal support member (18) by 2 identical screws (17). In order to move the leg, the bottom of the gear (11) is connected to a small, vertical member (19) which is connected to a small, horizontal member (20). This member (20) is attached to a steel rod (29), as shown in FIG. 5. A strong, durable, metal casing (21) covers this steel rod. The base of the steel rod (29) is connected to another horizontal support member (23) which is the beginning of the prosthetic foot. The prosthetic ankle joint (22) contains the support member (23), which is connected to the steel rod (29). The ankle joint (22) moves independently of the knee joint (9). The support member (23) is connected to two other diagonal support members (24). These members (24) hold two servo motors (16) that move the foot. A metal studding (25), as shown in FIG. 4, connects the two motors to a copper foot (30), as shown in FIG. 6. This copper foot is covered by a metal casing (26).

FIG. 5 shows an exploded view of the synergetic prosthesis (8) mentioned in FIG. 4. The exploded view of the knee joint (9) and the exploded view of the ankle joint (22) is shown. The steel shaft (28), the ball bearing (27), the screws (17), the vertical steel rod (29), the foot horizontal support member (23X the diagonal support members (24) that hold the motors (16), the studding (25), the copper foot (30), and the metal casing (26) can be clearly seen. In addition, (35) is a spacer used to connect the two horizontal support members (18) to prevent the gears from interlocking.

FIG. 6 depicts a block diagram of the control system (31). The power supply (32) provides power for the accelerometers (5) and the wireless transmitters (6). The two accelerometers (5) reads the angle of rotation for both the knee and the ankle and the wireless transmitters (6) sends the signals to the microcontroller (33). The microcontroller controls the two servo motors (16) based on the angles the accelerometer reads. Another power supply (32) provides power to the microcontroller (33) and the servo motors (16).

The invention claimed is:

1. A synergetic prosthesis system comprising:
a knee sleeve worn on the healthy leg
an ankle sleeve worn on the healthy leg
a prosthesis with knee joint assembly
a prosthesis with ankle joint assembly

2. The synergetic prosthesis system of claim 1, further comprising an accelerometer attached to the knee sleeve.

3. The synergetic prosthesis system of claim 1, further comprising an accelerometer attached to the ankle sleeve.

4. The synergetic prosthesis system of claim 2, wherein the accelerometer on the healthy leg measures the knee angle of rotation that is transmitted to the microcontroller.

5. The synergetic prosthesis system claim 3, wherein the accelerometer cm the healthy leg measures the ankle angle of rotation that is transmitted to the microcontroller.

6. The synergetic prosthesis system of claim 4 and claim 5, wherein the microcontroller receives the knee and the ankle angle of rotation signals from the healthy leg.

7. The synergetic prosthesis system of claim 11, wherein the microcontroller compares the knee and the ankle angle of rotation from the health leg to the preprogrammed normal human gait cycle knee and the ankle angle of rotation.

8. The synergetic prosthesis system of claim 7, wherein the micro-controller sends the output signals to servo motors to move the prosthetic knee and the ankle joints.
